(19) United States
(12) Patent Application Publication
(10) Pub. No.: US 2015/0182371 A1
(43) Pub. Date: Jul. 2, 2015

(54) BLOOD CIRCULATION ENHANCEMENT DEVICE

(71) Applicant: ELMER L OWENS, NEW YORK, NY (US)

(72) Inventor: ELMER L OWENS, NEW YORK, NY (US)

(21) Appl. No.: 14/146,616

(22) Filed: Jan. 2, 2014

(51) Int. Cl. A61F 5/41

(52) U.S. Cl. CPC A61F 5/41; A61F 2005/412

(57) ABSTRACT

The invention features a penile blood circulation enhancement device which enables a user to increase blood flow to the penis and enables the easy placement of a penile ring onto the user's penis to maintain the attained erection. One or more platforms on the body of the device enable the easy translation and placement of the penile ring onto the user's penis.

### FIELD OF THE INVENTION

The present invention relates, in general, to a device used to enhance and increase the blood flow to an individual's penis thereby increasing the length and/or width of the penis prior to and during coitus.

### BACKGROUND OF THE INVENTION

A growing number of men suffer from erectile dysfunction as a result of their advanced age and/or as a result of other health complications or ailments. In order to obtain an erection, some men use different devices to stimulate blood flow to the penis. However, maintaining penile blood flow and erections tends to be difficult. As such, there is a need for a device to help men obtain and maintain an erection. There is also a need for a device that enables the easy application of penile rings onto a penis which help keep the blood within the penis thereby maintaining the erection.

### SUMMARY OF THE INVENTION

The present invention provides a device that meets the stated needs above. An aspect of an embodiment of the invention provides a penile blood circulation enhancement device, which may include a cylindrical body contoured to receive a user's penis. The cylindrical body may have an opening which leads into a vacuum chamber designed to receive a user's penis. The device may also include a suction pump receptacle at an end of the cylindrical body opposite that of the opening. The suction pump receptacle may be adapted to receive or be connected to a suction feed from a suction pump. The enhancement device may further include a conical section which has its conical apex adjacent to the base of the suction pump receptacle, a cylindrical neck section immediately adjacent and beginning from the base of the conical section (which is opposite its conical apex) to the conical section of the cylindrical body and one or more platforms on the cylindrical body's surface. These platform(s) may extend along a certain length of the cylindrical body beginning from an end of the cylindrical neck section and extending to a location before the opening of the cylindrical body.

In an aspect of an embodiment of the present invention, the platform(s) may include a first gradient section, the first gradient section rising at a certain gradient from the end of the cylindrical neck section, a flat section following the first gradient section, and a second gradient section, having a downward gradient toward the location before the opening of the cylindrical body.

In another aspect of an embodiment of the present invention, the rising gradient of the first gradient section of the platform(s) may be between 0-30 degrees.

In another aspect of an embodiment of the present invention, the downward gradient of the second gradient section of the platform(s) may be less than 90 degrees.

In another aspect of an embodiment of the present invention, the cylindrical body may include a rim located at the cylindrical body's base. In one aspect, the rim may be adapted either physically or functionally to maintain a seal tight connection between the cylindrical body's base and the base of the user's penis.

In another aspect of an embodiment of the present invention, rim may be adapted to maintain the vacuum created within the vacuum chamber.

Additional aspects, objectives, features and advantages of the present invention will become apparent from the following description of the preferred embodiments with reference to the attached drawings.

### BRIEF DESCRIPTION OF THE DRAWINGS

FIG. 1 illustrates a side perspective view of a penile blood circulation enhancement device according to an aspect of an embodiment of the present invention.

FIG. 2 illustrates a bottom perspective view of a penile blood circulation enhancement device according to an aspect of an embodiment of the present invention.

FIG. 3 illustrates a side perspective view of a penile blood circulation enhancement device along with a penile ring according to an aspect of an embodiment of the present invention.

FIG. 4 illustrates a bottom perspective view of a penile blood circulation enhancement device along with a penile ring according to an aspect of an embodiment of the present invention.

FIG. 5 illustrates a bottom view of a penile blood circulation enhancement device according to an aspect of an embodiment of the present invention.

### DETAILED DESCRIPTION OF THE INVENTION

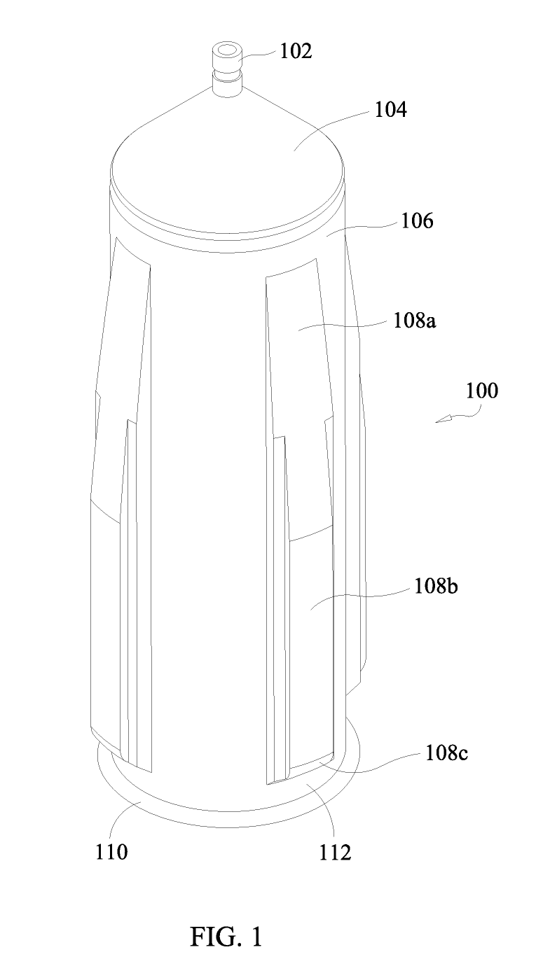

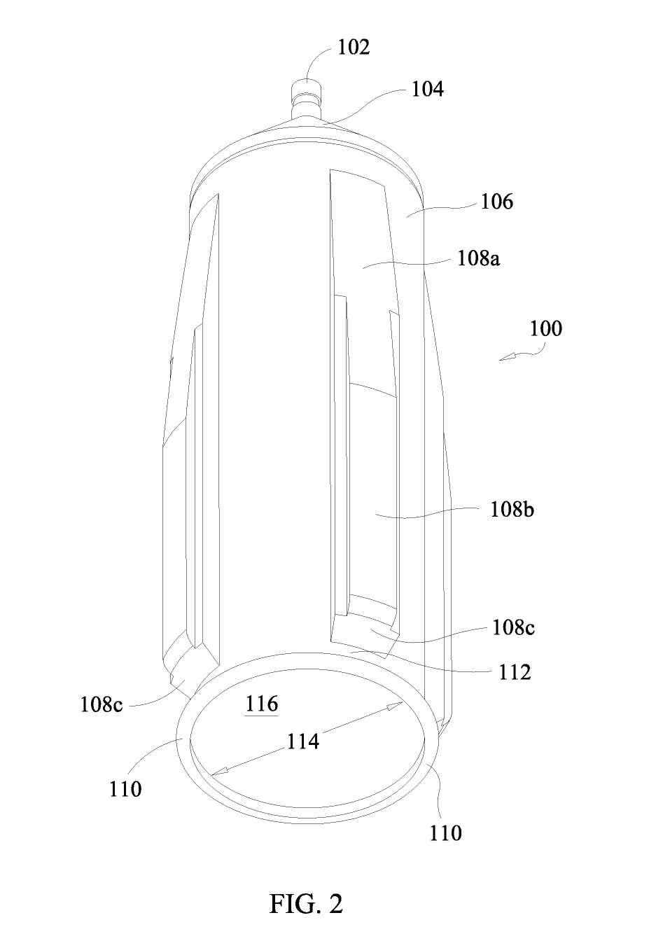

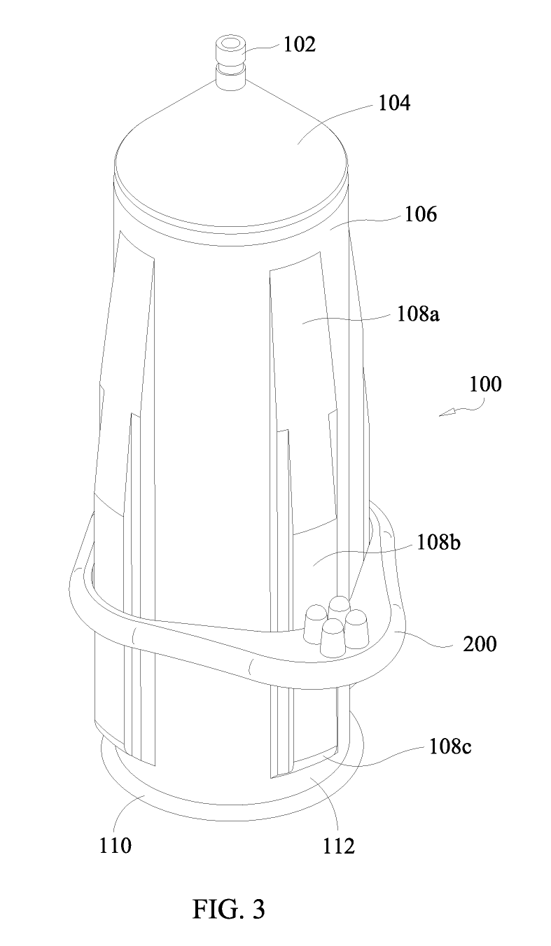

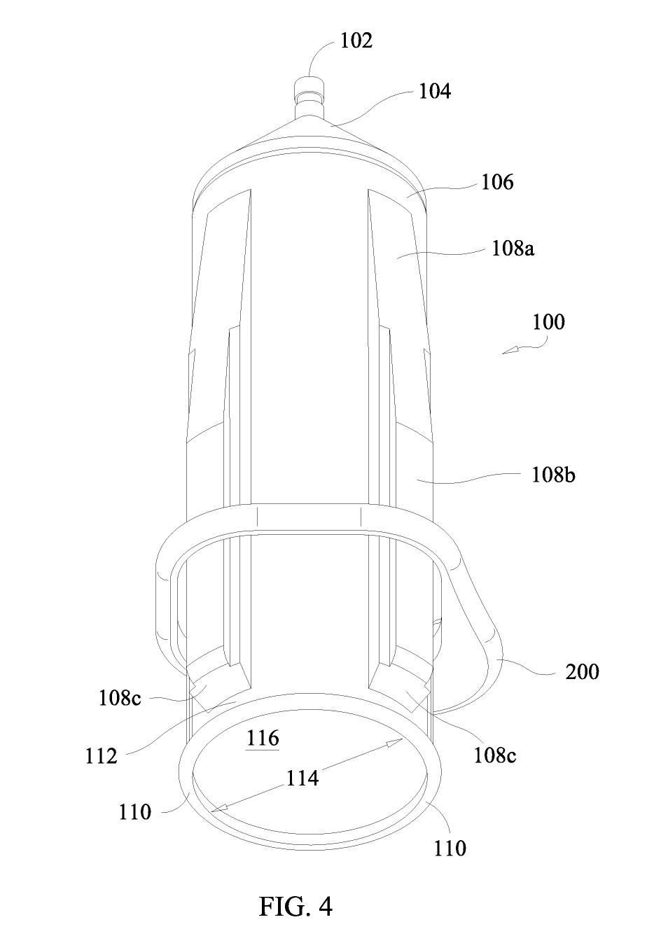

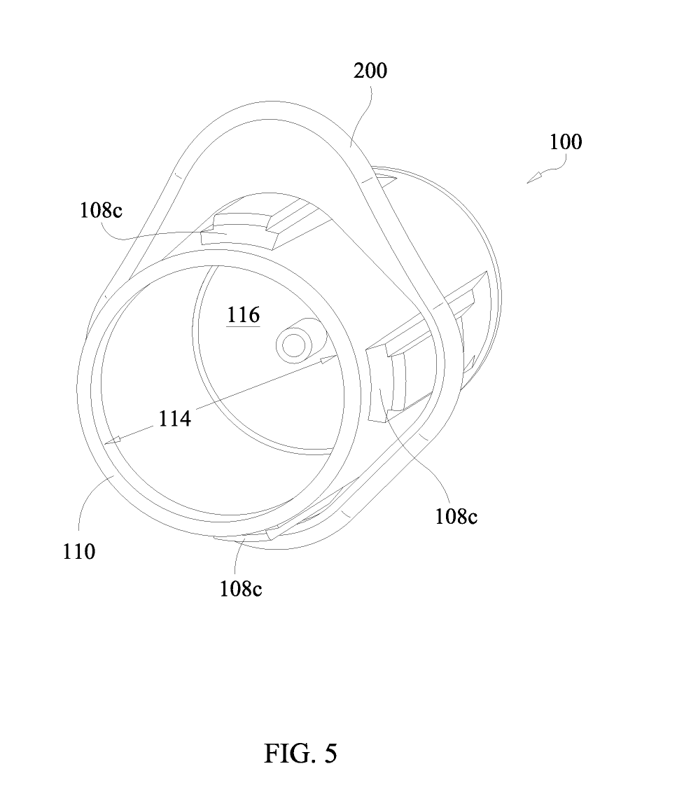

Referring now to FIGS. 1-4, side, bottom and respective perspective views of a penile blood circulation enhancement device 100 are shown. Enhancement device 100 is also shown, in FIGS. 3-5 with penile ring 200. Enhancement device 100, in one aspect of an embodiment of the present invention, may have an outer cylindrical body although other body configurations are possible. Enhancement device 100 may have an opening 114 which opens to vacuum chamber 116 which is contoured to receive a user's penis. At the end of enhancement device 100 opposite opening 114 is suction pump receptacle 102 which is internally connected (within the cylindrical body of enhancement device 100) with vacuum chamber 116. Suction pump receptacle 102 may be connected to a suction pump (not shown) which may be used to evacuate air from within vacuum chamber 116 in order to create a vacuum which results in increased blood flow to the user's penis, thereby enhancing the user's penile length and/or width. A user may place his penis into vacuum chamber 116 to begin the enhancement process (i.e. the air suction out of vacuum chamber 116 by way of air suction out of vacuum chamber 116 via suction pump receptacle 102) Immediately adjacent to the base of suction pump receptacle 102 is conical section 104 having both a conical apex and base. Conical section 104's conical apex may be adjacent to the base of suction pump receptacle 102 while its base spreads and extends away from suction pump receptacle 102 Immediately adjacent the base of conical section 104 is cylindrical neck section 106 which enables the placement and translation of penile ring 200 along the outer surface of enhancement device 100. Immediately following cylindrical neck section 106 are a number of platform(s) 108. In one aspect of an embodiment of the present invention, there may be one or more platforms, with each, in another aspect, having first gradient sections 108a. First gradient section 108a may have a rising gradient from the end of cylindrical neck section 106 at a rising gradient between 0 and 30 degrees. The functions of conical section 104, cylindrical neck section 106 and first gradient section 108a is to make it possible and easier for the user to slide penile ring 200 onto enhancement device 100.

Immediately following first gradient section 108a is flat section 108b of platform 108. In one aspect of an embodiment of the present invention, flat section 108b functions to enable the smooth translation of a penile ring down enhancement device 100 towards the user's penis in the direction as shown in FIG. 3. At the end of flat section 108b is second gradient section 108c which has a downward gradient down from flat section 108b. In one aspect of an embodiment of the present invention, second gradient 108c may have a downward gradient that may be less than 90 degrees. This downward gradient may be abrupt to ease and speed the placement of penile ring 200 off platforms 108 and onto the base of the user's penis. Second gradient section 108c ends with a space 112 between it (second gradient section 108c) and edge or rim 110 of opening 114 which leads into vacuum chamber 116. Space 112 enables and eases the placement of a penile ring onto the base of the user's penis. At the end of space 112 is edge or rim 110. Edge or rim 110 may be adapted to maintain a seal tight connection between the enhancement device 100's base and the base of the user's penis. In another aspect of an embodiment of the present invention, edge or rim 110 may comprise of a rubber lining (or equivalent structure) to ensure a seal tight connection between the cylindrical body's base and the base of the user's penis and to maintain the vacuum created within the vacuum chamber 116 while the vacuum/suction process is underway.

Operation of the blood circulation enhancement device 100 begins when a user places his penis within vacuum chamber 116 and places edge 110 of enhancement device 100 firmly around the base of his penis to ensure a proper seal. At first, the user would ensure that all pubic hair around the base of his penis has been shaved off to ensure that enhancement device 100 maintains a firm, seal-tight contact with the base of the user's penis. This also ensures that good suction is attained once the pumping begins. The user may also apply water-based lubricant around his penis. Once these steps have been undertaken, the user may plug a suction pump, penis pump or similar device (not shown) to suction pump plug receptacle 102. Then suction pump is then activated, either manually or mechanically, and air within vacuum chamber 116 is evacuated thereby creating a vacuum within vacuum chamber 116. As a result of the evacuation of air, blood flow to the user's penis is increased and the user's penile length and width are both enhanced. Once a desired width and/or length is attained, penis ring 200 may then be slid over conical section 104 of cylindrical body 100, onto and off platforms 108 and onto the base of the user's penis. The ease of penis ring 200's translation and placement is made possible by way of conical section 104, cylindrical neck section 106, platform 108's gradient (108a and 108c) and flat (108b) sections and space 112 which ease the sliding of the penis ring onto and off the platforms and eventually onto the base of the user's penis. Penis ring 200 then, by way of its constriction around the base of the user's penis, maintains the attained size by constricting blood flow out of the user's penis.

Although this present invention has been disclosed with reference to specific forms and embodiments, it will be evident that a great number of variations may be made without departing from the spirit and scope of the present invention. For example, equivalent elements may be substituted for those specifically disclosed and certain features of the present invention may be used independently of other features—all without departing from the present invention as defined in the appended claims

What is claimed is:

1. A penile blood circulation enhancement device, comprising:
a cylindrical body, contoured to receive a user's penis, having an opening at one end leading into a vacuum chamber;
a suction pump receptacle at an end of said cylindrical body opposite that of said opening, said suction pump receptacle being adapted to receive a suction feed from a suction pump, said suction plug receptacle being connected to said vacuum chamber;
a conical section of said cylindrical body, extending from and connected to said suction pump receptacle;
a cylindrical neck section immediately adjacent to said conical section of said cylindrical body; and
at least one platform on said cylindrical body's surface, said at least one platform extending along a certain length of said cylindrical body beginning from an end of said cylindrical neck section and extending to a location before said opening of said cylindrical body.

2. The penile blood circulation enhancement device according to claim 1, wherein said at least one platform comprises of a first gradient section, said first gradient section rising at a certain gradient from said end of said cylindrical neck section, a flat section following said first gradient section; and a second gradient section, having a downward gradient toward said location before said opening of said cylindrical body.

3. The penile blood circulation enhancement device according to claim 2, wherein said rising gradient of said first gradient section of said at least one platform is between 0-30 degrees.

4. The penile blood circulation enhancement device according to claim 2, wherein said downward gradient of said second gradient section of said at least one platform is less than 90 degrees.

5. The penile blood circulation enhancement device of claim 1 further comprising a rim located at said cylindrical body's base.

6. The penile blood circulation enhancement device of claim 5, wherein said rim is adapted to maintain a seal tight connection between said cylindrical body's base and the base of said user's penis.

7. The penile blood circulation enhancement device of claim 5, wherein said rim is adapted to maintain vacuum created within said vacuum chamber.
